(19) United States
(12) Patent Application Publication
(10) Pub. No.: US 2025/0213160 A1
(43) Pub. Date: Jul. 3, 2025

(54) Flexible Microelectrode Arrays

(71) Applicant: Nikon Corporation, Tokyo (JP)

(72) Inventors: Wan Qin, Oro Valley, AZ (US); Fardad Ali Hashemi, Menlo Park, CA (US)

(73) Assignee: Nikon Corporation, Tokyo (JP)

(21) Appl. No.: 18/853,415

(22) PCT Filed: Apr. 4, 2022
(86) PCT No.: PCT/US22/23291

§ 371 Date: Oct. 2, 2024

(51) Int. Cl. A61B 5/263; A61B 5/268; A61N 1/04; B81B 1/00; B81C 1/00

(52) U.S. Cl. CPC A61B 5/263; A61B 5/268; A61N 1/0456; B81B 1/008; B81C 1/00111; A61B 2562/12; B81B 2203/0361; B81B 2203/04; B81C 2201/032

(57) ABSTRACT

The problems of high costs and lack of flexibility in microelectrode arrays (MEAs) is addressed by the inexpensive flexible MEA systems and methods for manufacturing them presented herein. The MEA systems described herein are generally formed from a flexible substrate such as polydimethylsiloxane (PDMS). The flexible substrate generally comprises a series of wells and channels patterned therein. The wells and channels are filled with a conductive flexible material such as a mixture of PDMS and carbon nanotubes (CNTs) to form sets of microelectrodes, microelectrode leads, and contact pads therein. The resulting MEA systems may be substantially more flexible and less expensive than prior MEA systems. The MEA systems presented herein may be manufactured using a variety of soft lithography techniques described herein.

### BACKGROUND OF THE INVENTION

Microelectrode arrays (MEAs) utilize arrays of microelectrodes to obtain signals from neurons or to transmit signals to neurons (e.g., to stimulate neurons to fire in certain ways). Thus, MEAs essentially serve as interfaces that connect neurons to electronic circuitry. Prior MEAs have typically used hard, non-flexible substrates such as silicon or glass. This limits their applicability to curved surfaces, such as an animal's head. Moreover, these prior MEAs have typically been constructed using traditional silicon and/or glass microfabrication processes (such as metal deposition, lithography, and etching), making such prior MEAs relatively expensive. Accordingly, presented herein are inexpensive flexible MEA systems and methods for manufacturing them.

### BRIEF DESCRIPTION OF THE DRAWINGS

Various embodiments of the invention are disclosed in the following detailed description and the accompanying drawings.

FIG. 1A shows a schematic depicting a cross-sectional view of an exemplary flexible microelectrode array (MEA) system.

FIG. 1B shows a schematic depicting a top view of the exemplary flexible MEA system of FIG. 1A.

FIG. 2 shows a flowchart for a first method for manufacturing the exemplary flexible MEA system of FIGS. 1A and 1B.

FIG. 3 shows a flowchart for a second method for manufacturing the exemplary flexible MEA system of FIGS. 1A and 1B.

FIG. 4 shows a flowchart for a third method for manufacturing the exemplary flexible MEA system of FIGS. 1A and 1B.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term “processor” refers to one or more devices, circuits, and/or processing cores configured to process data, such as computer program instructions.

A detailed description of one or more embodiments of the invention is provided below along with accompanying Figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

As used herein, the term “or” shall convey both disjunctive and conjunctive meanings. For instance, the phrase “A or B” shall be interpreted to include element A alone, element B alone, and the combination of elements A and B.

In the Figures (also “FIGs.”), like numbers shall refer to like elements.

Microelectrode arrays (MEAs) utilize arrays of microelectrodes to obtain signals from neurons or to transmit signals to neurons (e.g., to stimulate neurons to fire in certain ways). Thus, MEAs essentially serve as interfaces that connect neurons to electronic circuitry.

Prior MEAs have typically used hard, non-flexible substrates such as silicon or glass. This limits their applicability to curved surfaces, such as an animal's head. Moreover, these prior MEAs have typically been constructed using traditional silicon and/or glass microfabrication processes (such as metal deposition, lithography, and etching), making such prior MEAs relatively expensive.

Accordingly, the problems of high costs and lack of flexibility in MEAs is addressed by the inexpensive flexible MEA systems and methods for manufacturing them presented herein. The MEA systems described herein are generally formed from a flexible substrate such as polydimethylsiloxane (PDMS). The flexible substrate generally comprises a series of wells and channels patterned therein. The wells and channels are filled with a conductive flexible material such as a mixture of PDMS and carbon nanotubes (CNTs) to form sets of microelectrodes, microelectrode leads, and contact pads therein. The resulting MEA systems may be substantially more flexible and less expensive than prior MEA systems. The MEA systems presented herein may be manufactured using a variety of soft lithography techniques described herein.

A flexible MEA system is disclosed herein. The system generally comprises: a flexible substrate comprising: a plurality of microelectrode wells patterned therein; a plurality of microelectrode lead channels patterned therein; and a plurality of contact pad wells patterned therein; a plurality of microelectrodes; a plurality of microelectrode leads; and a plurality of contact pads. In some embodiments, each microelectrode lead channel is coupled to a microelectrode well of the plurality of microelectrode wells. In some embodiments, each contact pad well is coupled to a microelectrode lead channel of the plurality of microelectrode lead channels. In some embodiments, each microelectrode is located within a microelectrode well of the plurality of microelectrode wells. In some embodiments, each microelectrode comprises a first flexible electrically conductive material. In some embodiments, each microelectrode lead is located within a microelectrode lead channel of the plurality of microelectrode lead channels. In some embodiments, each microelectrode lead comprises a second flexible electrically conductive material. In some embodiments, each microelectrode lead is electrically coupled to a microelectrode of the plurality of microelectrodes. In some embodiments, each contact pad is located within a contact pad well of the plurality of contact pad wells. In some embodiments, each contact pad comprises a third flexible electrically conductive material. In some embodiments, each contact pad is electrically coupled to a microelectrode lead of the plurality of microelectrode leads. In some embodiments, the flexible substrate is selected from the group consisting of: silicone, polydimethylsiloxane (PDMS), polyimide, and any combination thereof. In some embodiments, the first, second, and third flexible electrically conductive materials are the same. In some embodiments, the first, second, and third flexible electrically conductive materials are different. In some embodiments, the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

A first method for manufacturing a flexible MEA system is disclosed herein. The method generally comprises: lithographically patterning a photoresist on a wafer; coating and curing a first layer of PDMS to cover the wafer and the photoresist; removing the first layer of PDMS from the wafer and the photoresist to thereby expose a flexible substrate comprising: a plurality of microelectrode wells patterned therein; a plurality of microelectrode lead channels patterned therein; and a plurality of contact pad wells patterned therein; filling the plurality of microelectrode wells with a first flexible electrically conductive material to thereby form a plurality of microelectrodes; filling the plurality of microelectrode lead channels with a second flexible electrically conductive material to thereby form a plurality of microelectrode leads; filling the plurality of contact pad wells with a third flexible electrically conductive material to thereby form a plurality of contact pads; and coating and curing a second layer of PDMS on the flexible substrate. In some embodiments, each microelectrode lead channel is coupled to a microelectrode well of the plurality of microelectrode wells. In some embodiments, each contact pad well is coupled to a microelectrode lead channel of the plurality of microelectrode lead channels. In some embodiments, each microelectrode is located within a microelectrode well of the plurality of microelectrode wells. In some embodiments, each microelectrode lead is located within a microelectrode lead channel of the plurality of microelectrode channels. In some embodiments, each microelectrode lead is electrically coupled to a microelectrode of the plurality of microelectrodes. In some embodiments, each contact pad is located within a contact pad well of the plurality of contact pad wells. In some embodiments, each contact pad is electrically coupled to a microelectrode lead of the plurality of microelectrode leads. In some embodiments, the first, second, and third flexible electrically conductive materials are the same. In some embodiments, the first, second, and third flexible electrically conductive materials are different. In some embodiments, the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof. In some embodiments, the method further comprises etching the first layer of PDMS.

A second method for manufacturing a flexible MEA system is disclosed herein. The method generally comprises: patterning a first layer of photoresist on a wafer; coating and curing a first layer of PDMS to cover the wafer and the first layer of photoresist; lithographically patterning a second layer of photoresist on the first layer of PDMS; selectively etching the first layer of PDMS through the second layer of photoresist; removing the second layer of photoresist from the first layer of PDMS; removing the first layer of PDMS from the wafer and the first layer of photoresist to thereby expose a flexible substrate comprising: a plurality of microelectrode wells patterned therein; a plurality of microelectrode lead channels patterned therein; and a plurality of contact pad wells patterned therein; filling the plurality of microelectrode wells with a first flexible electrically conductive material to thereby form a plurality of microelectrodes; filling the plurality of microelectrode lead channels with a second flexible electrically conductive material to thereby form a plurality of microelectrode leads; filling the plurality of contact pad wells with a third flexible electrically conductive material to thereby form a plurality of contact pads; and coating and curing a second layer of PDMS on the flexible substrate. In some embodiments, each microelectrode lead channel is coupled to a microelectrode well of the plurality of microelectrode wells. In some embodiments, each contact pad well is coupled to a microelectrode lead channel of the plurality of microelectrode lead channels. In some embodiments, each microelectrode is located within a microelectrode well of the plurality of microelectrode wells. In some embodiments, each microelectrode lead is located within a microelectrode lead channel of the plurality of microelectrode channels. In some embodiments, each microelectrode lead is electrically coupled to a microelectrode of the plurality of microelectrodes. In some embodiments, each contact pad is located within a contact pad well of the plurality of contact pad wells. In some embodiments, each contact pad is electrically coupled to a microelectrode lead of the plurality of microelectrode leads. In some embodiments, the first, second, and third flexible electrically conductive materials are the same. In some embodiments, the first, second, and third flexible electrically conductive materials are different. In some embodiments, the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof. In some embodiments, the method further comprises etching the second layer of PDMS.

A third method for manufacturing a flexible MEA system is disclosed herein. The method generally comprises: lithographically patterning a photoresist on a wafer; coating and curing a first layer of PDMS to cover the wafer and the photoresist; removing the first layer of PDMS from the wafer and the photoresist to thereby expose a first flexible substrate comprising a plurality of microelectrode lead channels patterned therein; punching a plurality of microelectrode wells into the first layer of PDMS; punching a plurality of contact pad wells into the first layer of PDMS; adhering a second flexible substrate to the first layer of PDMS; and flowing a flexible electrically conductive material into the plurality of microelectrode wells or into the plurality of contact pad wells, thereby filling the plurality of microelectrode wells, the plurality of microelectrode lead channels, and the plurality of contact pad wells with the flexible electrically conductive material to thereby form: a plurality of microelectrodes located between the first and second flexible substrates; a plurality of microelectrode leads located between the first and second flexible substrates; and a plurality of contact pads located between the first and second flexible substrates. In some embodiments, each microelectrode is coupled to a first end of a microelectrode lead channel of the plurality of microelectrode lead channels. In some embodiments, each contact pad well is coupled to a second end of a microelectrode lead channel of the plurality of microelectrode lead channels. In some embodiments, each microelectrode is located within a microelectrode well of the plurality of microelectrode wells. In some embodiments, each microelectrode lead is located within a microelectrode lead channel of the plurality of microelectrode channels. In some embodiments, each microelectrode lead is electrically coupled to a microelectrode of the plurality of microelectrodes. In some embodiments, each contact pad is located within a contact pad well of the plurality of contact pad wells. In some embodiments, each contact pad is electrically coupled to a microelectrode lead of the plurality of microelectrode leads. In some embodiments, the second flexible substrate is selected from the group consisting of: silicone, PDMS, polyimide, and any combination thereof. In some embodiments, the flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

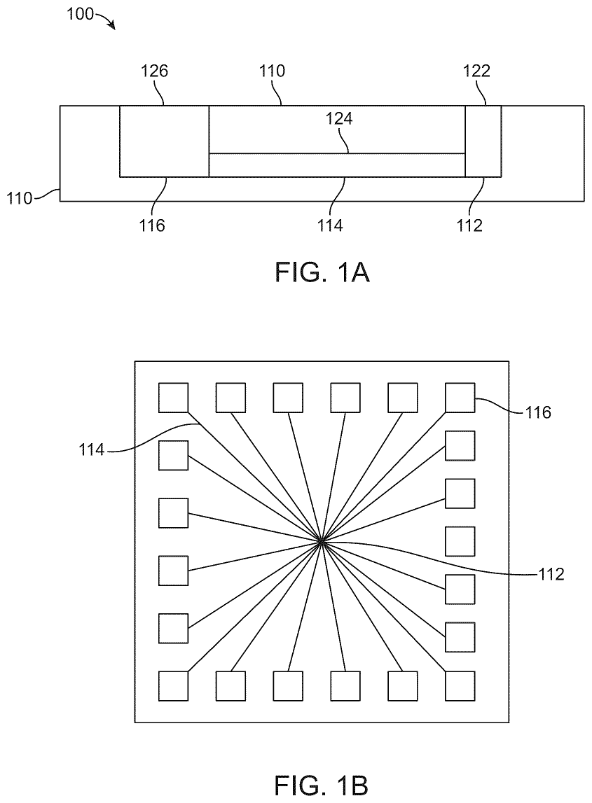

FIG. 1A shows a schematic depicting a cross-sectional view of an exemplary flexible MEA system 100. In the example shown, the system comprises a flexible substrate 110. In some embodiments, the flexible substrate is selected from the group consisting of: polyimide, silicone, PDMS, polyimide, and any combination thereof.

In the example shown, the flexible substrate 110 comprises a plurality of microelectrode wells 112 patterned therein, a plurality of microelectrode lead channels 114 patterned therein, and a plurality of contact pad wells 116 patterned therein. In some embodiments, each microelectrode lead channel is coupled (for instance, mechanically or fluidically coupled) to a microelectrode well of the plurality of microelectrodes. In some embodiments, each contact pad well is coupled (for instance, mechanically or fluidically coupled) to a microelectrode lead channel of the plurality of microelectrode lead channels.

In some embodiments, each microelectrode well, each microelectrode lead channel, or each contact pad well has a depth of at least about 100 nanometers (nm), 200 nm, 300 nm, 400 nm, 500 nm, 600 nm, 700 nm, 800 nm, 900 nm, 1 micrometer (μm), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. In some embodiments, each microelectrode well, each microelectrode lead channel, or each contact pad well has a depth of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 900 nm, 800 nm, 700 nm, 600 nm, 500 nm, 400 nm, 300 nm, 200 nm, 100 nm, or less. In some embodiments, each microelectrode well, each microelectrode lead channel, or each contact pad well has a depth that is within a range defined by any two of the preceding values.

In some embodiments, each microelectrode well, each microelectrode lead channel, or each contact pad well has a width of at least about 100 nm, 200 nm, 300 nm, 400nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 millimeter (mm), 2 mm, 3 mm, 4 mm, 5 mm, 6 mm, 7 mm, 8 mm, 9 mm, 10 mm, or more. In some embodiments, each microelectrode well, each microelectrode lead channel, or each contact pad well has a width of at most about 10 mm, 9 mm, 8 mm, 7 mm, 6 mm, 5 mm, 4 mm, 3mm, 2 mm, 1 mm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 um, 6 μm, 5 μm, 4 μm, 3 μm, 2 μm, 1 μm, 900 nm, 800 nm, 700 nm, 600 nm, 500 nm, 400 nm, 300 nm, 200 nm, 100 nm, or less. In some embodiments, each microelectrode well, each microelectrode lead channel, or each contact pad well has a width that is within a range defined by any two of the preceding values.

In some embodiments, each microelectrode well, each microelectrode lead channel, or each contact pad well has a length of at least about 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, 10 mm, 20 mm, 30 mm, 40 mm, 50 mm, 60 mm, 70 mm, 80 mm, 90 mm, 100 mm, or more. In some embodiments, each microelectrode well, each microelectrode lead channel, or each contact pad well has a length of at most about 100 mm, 90 mm, 80 mm, 70 mm, 60 mm, 50 mm, 40 mm, 30 mm, 20 mm, 10 mm, 9 mm, 8 mm, 7 mm, 6 mm, 5 mm, 4 mm, 3 mm, 2 mm, 1 mm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μ, 9 μ, 8 μ, 7 μ, 6 μ, 5 μ, 4 μ, 3 μm, 2 μm, 1 μm, 900 nm, 800 nm, 700 nm, 600 nm, 500 nm, 400 nm, 300 nm, 200 nm, 100 nm, or less. In some embodiments, each microelectrode well, each microelectrode lead channel, or each contact pad well has a length that is within a range defined by any two of the preceding values.

Although depicted as comprising a single microelectrode well, a single microelectrode lead channel, and a single contact pad well in FIG. 1A, the flexible substrate may comprise any number of microelectrode wells, any number of microelectrode lead channels, and any number of contact pad wells. In some embodiments, the flexible substrate comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or more microelectrode wells, microelectrode lead channels, or contact pad wells. In some embodiments, the flexible substrate comprises at most about 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 microelectrode wells, microelectrode lead channels, or contact pad wells. In some embodiments, the flexible substrate comprises a number of microelectrode wells, microelectrode lead channels, or contact pad wells that is within a range defined by any two of the preceding values.

In the example shown, the system 100 comprises a plurality of microelectrodes 122. In some embodiments, each microelectrode is located within a microelectrode well of the plurality of microelectrode wells. In some embodiments, each microelectrode comprises a first flexible electrically conductive material. In some embodiments, the first electrically conductive material is selected from the group consisting of: a mixture of silicone and CNTs, a mixture of PDMS and CNTs, CNT ink, a metallic ink, silver ink, gold ink, aluminum ink, copper ink, and any combination thereof.

In the example shown, the system 100 comprises a plurality of microelectrode leads 124. In some embodiments, each microelectrode lead is located within a microelectrode lead channel of the plurality of microelectrode lead channels. In some embodiments, each microelectrode lead is electrically coupled to a microelectrode of the plurality of microelectrodes. In some embodiments, each microelectrode lead comprises a second flexible electrically conductive material. In some embodiments, the second flexible electrically conductive material is the same as the first flexible electrically conductive material. In some embodiments, the second flexible electrically conductive material is different from the first flexible electrically conductive material. In some embodiments, the second electrically conductive material is selected from the group consisting of: a mixture of silicone and CNTs, a mixture of PDMS and CNTs, CNT ink, a metallic ink, silver ink, gold ink, aluminum ink, copper ink, and any combination thereof.

In the example shown, the system 100 comprises a plurality of contact pads 126. In some embodiments, each contact pad is located within a contact pad well of the plurality of contact pad wells. In some embodiments, each contact pad is electrically coupled to a microelectrode lead of the plurality of microelectrode leads. In some embodiments, each contact pad comprises a third flexible electrically conductive material. In some embodiments, the third flexible electrically conductive material is the same as the first flexible electrically conductive material or the second flexible electrically conductive material. In some embodiments, the third flexible electrically conductive material is different from the first flexible electrically conductive material or the second flexible electrically conductive material. In some embodiments, the third electrically conductive material is selected from the group consisting of: a mixture of silicone and CNTs, a mixture of PDMS and CNTs, CNT ink, a metallic ink, silver ink, gold ink, aluminum ink, copper ink, and any combination thereof.

In some embodiments, each microelectrode is configured to electrically couple to a neuron or group of neurons (for instance, at least about 1, 2, 3, 4, 5, 6, 7, 8, 9, 10, or more neurons, at most about 10, 9, 8, 7, 6, 5, 4, 3, 2, or 1 neurons, or a number of neurons that is within a range defined by any two of the preceding values) and to obtain signals therefrom or transmit signals thereto. In some embodiments, each microelectrode lead and each contact pad is configured to obtain the neural signals from or transmit the neural signals to the microelectrode to which it is electrically coupled. In some embodiments, each contact pad is configured to interface with a controller configured to process the neural signals obtained by or to generate the neural signals transmitted by the microelectrode to which it is electrically coupled.

In some embodiments, each microelectrode, each microelectrode lead, or each contact pad has a depth of at least about 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 um, 500 μm, 600 μm, 700 μm, 800 μm, 900 μm, 1,000 μm, or more. In some embodiments, each microelectrode, each microelectrode lead, or each contact pad has a depth of at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μ, 4 μ, 3 μm, 2 μm, 1 μm, 900 nm, 800 nm, 700 nm, 600 nm, 500 nm, 400 nm, 300 nm, 200 nm, 100 nm, or less. In some embodiments, each microelectrode, each microelectrode lead, or each contact pad has a depth that is within a range defined by any two of the preceding values.

In some embodiments, each microelectrode, each microelectrode lead, or each contact pad has a width of at least about 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 um, 20 μm, 30 μm, 40 μm, 50 μm, 60 μm, 70 μm, 80 μm, 90 μm, 100 μm, 200 μm, 300 μm, 400μm, 500 μm, 600 μm, 700 μm, 800 μm, 900 μm, 1 mm, 2 mm, 3 mm, 4 mm, 5 mm, 6 mm, 7mm, 8 mm, 9 mm, 10 mm, or more. In some embodiments, each microelectrode, each microelectrode lead, or each contact pad has a width of at most about 10 mm, 9 mm, 8 mm, 7mm, 6 mm, 5 mm, 4 mm, 3 mm, 2 mm, 1 mm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20μm, 10 μ, 9 μm, 8 μm, 7 μ, 6 μm, 5 μ, 4 μ, 3 μm, 2 μ, 1 μm, 900 nm, 800 nm, 700nm, 600 nm, 500 nm, 400 nm, 300 nm, 200 nm, 100 nm, or less. In some embodiments, each microelectrode, each microelectrode lead, or each contact pad has a width that is within a range defined by any two of the preceding values.

In some embodiments, each microelectrode, each microelectrode lead, or each contact pad has a length of at least about 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400μm, 500 μm, 600 μm, 700 μm, 800 μm, 900 μm, 1 mm, 2 mm, 3 mm, 4 mm, 5 mm, 6 mm, 7mm, 8 mm, 9 mm, 10 mm, 20 mm, 30 mm, 40 mm, 50 mm, 60 mm, 70 mm, 80 mm, 90 mm, 100 mm, or more. In some embodiments, each microelectrode, each microelectrode lead, or each contact pad has a length of at most about 100 mm, 90 mm, 80 mm, 70 mm, 60 mm, 50mm, 40 mm, 30 mm, 20 mm, 10 mm, 9 mm, 8 mm, 7 mm, 6 mm, 5 mm, 4 mm, 3 mm, 2 mm, 1 mm, 900 μm, 800 μm, 700 μm, 600 μm, 500 μm, 400 μm, 300 μm, 200 μm, 100 μm, 90μm, 80 μm, 70 μm, 60 μm, 50 μm, 40 μm, 30 μm, 20 μm, 10 μm, 9 μm, 8 μm, 7 μm, 6 μm, 5μm, 4 μm, 3 μm, 2 μm, 1 μm, 900 nm, 800 nm, 700 nm, 600 nm, 500 nm, 400 nm, 300 nm, 200 nm, 100 nm, or less. In some embodiments, each microelectrode, each microelectrode lead, or each contact pad has a length that is within a range defined by any two of the preceding values.

Although depicted as comprising a single microelectrode, a single microelectrode lead, and a single contact pad in FIG. 1A, the system 100 may comprise any number of microelectrodes, any number of microelectrode leads, and any number of contact pads. In some embodiments, the system comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or more microelectrodes, microelectrode leads, or contact pads. In some embodiments, the system comprises at most about 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 microelectrodes, microelectrode leads, or contact pads. In some embodiments, the system comprises a number of microelectrodes, microelectrode leads, or contact pads that is within a range defined by any two of the preceding values.

FIG. 1B shows a schematic depicting a top view of the exemplary flexible MEA system of FIG. 1A. In the example shown, the plurality of microelectrodes 122 are arranged in an array within the flexible substrate 110. In some embodiments, the plurality of microelectrodes are spaced apart by a distance of at least about 1 μm, 2 μm, 3 μm, 4 μm, 5μm, 6 μm, 7 μm, 8 μm, 9 μm, 10 μm, 20 μm, 30 μm, 40 μm, 50 μm, 60 μm, 70 μm, 80 μm, 90μm, 100 μm, 200 μm, 300 μm, 400 μm, 500 μm, 600 μm, 700 μm, 800 μm, 900 μm, 1 mm, or more. In some embodiments, the plurality of microelectrodes are spaced apart by a distance of at most about 1 mm, 900 μm, 800 μm, 700 μm, 600 μm, 500 μm, 400 μm, 300 μm, 200μm, 100 μm, 90 μm, 80 μm, 70 μm, 60 μm, 50 μm, 40 μm, 30 μm, 20 μm, 10 μm, 9 μm, 8μm, 7 μm, 6 μm, 5 μm, 4 μm, 3 μm, 2 μm, 1 μm, or less. In some embodiments, the plurality of microelectrodes are spaced apart by a distance that is within a range defined by any two of the preceding values.

In the example shown, the plurality of microelectrode leads 124 and the plurality of contact pads 126 are arranged in an array around the plurality of microelectrodes.

The arrangement of microelectrodes, microelectrode leads, and contact pads depicted in FIGS. 1A and 1B is illustrative only. One having skill in the art will recognize that other arrangements are possible and within the scope of this disclosure.

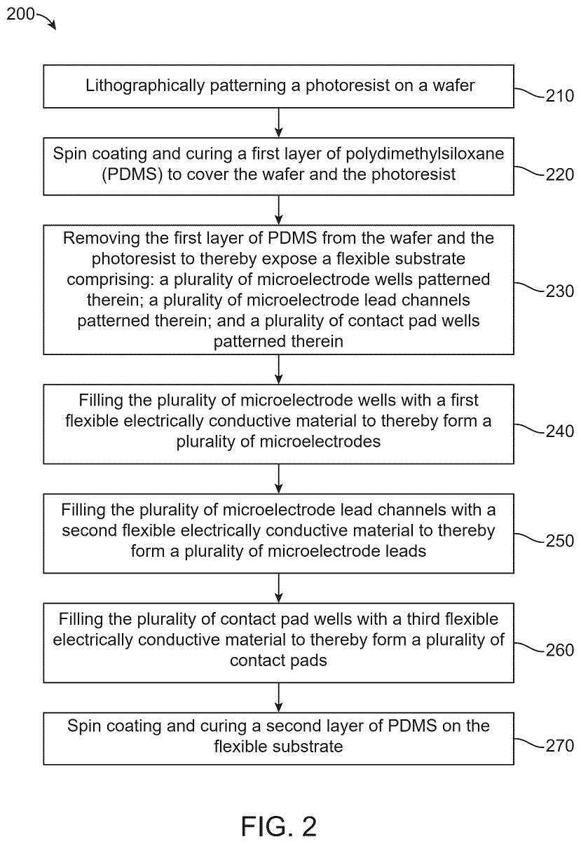

FIG. 2 shows a flowchart for a first method 200 for manufacturing the exemplary flexible MEA system 100 of FIGS. 1A and 1B. At 210, a photoresist is lithographically patterned on a wafer (such as a silicon wafer). In some embodiments, the photoresist comprises SU-8 photoresist. In some embodiments, the photoresist is patterned to form a negative mold of the microelectrode wells, microelectrode lead channels, and contact pad wells described herein.

At 220, a first layer of PDMS is coated and cured to cover the wafer and the photoresist. In some embodiments, the first layer of PDMS is coated via spin coating.

At 230, the first layer of PDMS is removed from the wafer and the photoresist to thereby expose a flexible substrate. In some embodiments, the flexible substrate comprises any flexible substrate described herein with respect to FIGS. 1A and 1B. In some embodiments, the flexible substrate comprises any plurality of microelectrode wells, plurality of microelectrode lead channels, and plurality of contact pad wells described herein with respect to FIGS. 1A and 1B. In some embodiments, each microelectrode lead channel is coupled to a microelectrode well of the plurality of microelectrode wells. In some embodiments, each contact pad well is coupled to a microelectrode lead channel of the plurality of microelectrode lead channels.

At 240, the plurality of microelectrode wells is filled with a first flexible electrically conductive material to thereby form a plurality of microelectrodes. In some embodiments, the plurality of microelectrodes comprise any plurality of microelectrodes described herein with respect to FIGS. 1A and 1B. In some embodiments, the first flexible electrically conductive material comprises any first flexible electrically conductive material described herein with respect to FIGS. 1A and 1B. In some embodiments, each microelectrode is located within a microelectrode well of the plurality of microelectrode wells.

At 250, the plurality of microelectrode lead channels are filled with a second flexible electrically conductive material to thereby form a plurality of microelectrode leads. In some embodiments, the plurality of microelectrode leads comprise any plurality of microelectrode leads described herein with respect to FIGS. 1A and 1B. In some embodiments, the second flexible electrically conductive material comprises any second flexible electrically conductive material described herein with respect to FIGS. 1A and 1B. In some embodiments, each microelectrode lead is located within a microelectrode lead channel of the plurality of microelectrode channels. In some embodiments, each microelectrode lead is electrically coupled to a microelectrode of the plurality of microelectrodes.

At 260, the plurality of contact pad wells are filled with a third flexible electrically conductive material to thereby form a plurality of contact pads. In some embodiments, the plurality of contact pads comprise any plurality of contact pads described herein with respect to FIGS. 1A and 1B. In some embodiments, the third flexible electrically conductive material comprises any third flexible electrically conductive material described herein with respect to FIGS. 1A and 1B. In some embodiments, each contact pad is located within a contact pad well of the plurality of contact pad wells. In some embodiments, each contact pad is electrically coupled to a microelectrode lead of the plurality of microelectrode leads.

At 270, a second layer of PDMS is coated and cured on the flexible substrate. In some embodiments, the second layer of PDMS is coated via spin coating. In some embodiments, the second layer of PDMS electrically insulates each microelectrode lead, as well as one side of each microelectrode and one side of each contact pad, from the environment. In some embodiments, the electrical insulation of the second layer of PDMS ensures that the neural signals are not interfered with by undesired contact with the microelectrode leads or the “inactive” side of the contact pads or microelectrode leads.

In some embodiments, the method 200 further comprises etching the first layer of PDMS. In some embodiments, the first layer of PDMS is etched using a reactive ion etching (RIE) process. In some embodiments, the RIE process is a sulfur hexafluoride (SF6) and oxygen (O2) RIE process. In some embodiments, etching the first layer of PDMS exposes the plurality of microelectrodes and the plurality of contact pads to the environment, while leaving the plurality of microelectrode leads electrically insulated from the environment. In some embodiments, this allows the plurality of microelectrodes to be electrically coupled to a single neuron or group of neurons and the plurality of contact pads to be electrically coupled to electronics that receive and process neural signals from the plurality of microelectrodes or that transmit neural signals to the plurality of microelectrodes.

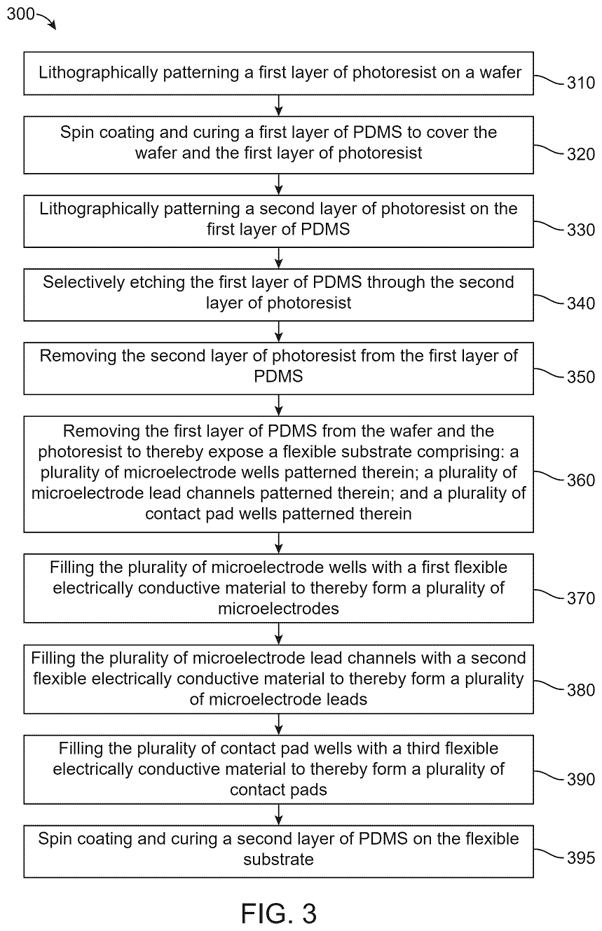

FIG. 3 shows a flowchart for a second method for manufacturing the exemplary flexible MEA system of FIGS. 1A and 1B. At 310, a first layer of photoresist is lithographically patterned on a wafer (such as a silicon wafer). In some embodiments, the first layer of photoresist comprises SU-8 photoresist. In some embodiments, the first layer of photoresist is patterned to form a partial negative mold of a portion of the microelectrode wells, the microelectrode lead channels, and a portion of the contact pad wells described herein.

At 320, a first layer of PDMS is coated and cured to cover the wafer and the first layer of photoresist. In some embodiments, the first layer of PDMS is coated via spin coating.

At 330, a second layer of photoresist is lithographically patterned on the first layer of PDMS. In some embodiments, the second layer of photoresist comprises S1813 positive photoresist.

At 340, the first layer of PDMS is selectively etched through the second layer of photoresist. In some embodiments, the first layer of PDMS is etched using a RIE process. In some embodiments, the RIE process is a SF₆ and O₂ RIE process.

At 350, the second layer of photoresist is removed from the first layer of PDMS. In some embodiments, the second layer of photoresist is removed by washing with isopropyl alcohol (IPA), by washing with acetone, by O₂ plasma etching, or by any combination thereof.

At 360, the first layer of PDMS is removed from the wafer and the first layer of photoresist to thereby expose a flexible substrate. In some embodiments, the flexible substrate comprises any flexible substrate described herein with respect to FIGS. 1A and 1B. In some embodiments, the flexible substrate comprises any plurality of microelectrode wells, plurality of microelectrode lead channels, and plurality of contact pad wells described herein with respect to FIGS. 1A and 1B. In some embodiments, each microelectrode lead channel is coupled to a microelectrode well of the plurality of microelectrode wells. In some embodiments, each contact pad well is coupled to a microelectrode lead channel of the plurality of microelectrode lead channels.

At 370, the plurality of microelectrode wells is filled with a first flexible electrically conductive material to thereby form a plurality of microelectrodes. In some embodiments, the plurality of microelectrodes comprise any plurality of microelectrodes described herein with respect to FIGS. 1A and 1B. In some embodiments, the first flexible electrically conductive material comprises any first flexible electrically conductive material described herein with respect to FIGS. 1A and 1B. In some embodiments, each microelectrode is located within a microelectrode well of the plurality of microelectrode wells.

At 380, the plurality of microelectrode lead channels are filled with a second flexible electrically conductive material to thereby form a plurality of microelectrode leads. In some embodiments, the plurality of microelectrode leads comprise any plurality of microelectrode leads described herein with respect to FIGS. 1A and 1B. In some embodiments, the second flexible electrically conductive material comprises any second flexible electrically conductive material described herein with respect to FIGS. 1A and 1B. In some embodiments, each microelectrode lead is located within a microelectrode lead channel of the plurality of microelectrode channels. In some embodiments, each microelectrode lead is electrically coupled to a microelectrode of the plurality of microelectrodes.

At 390, the plurality of contact pad wells are filled with a third flexible electrically conductive material to thereby form a plurality of contact pads. In some embodiments, the plurality of contact pads comprise any plurality of contact pads described herein with respect to FIGS. 1A and 1B. In some embodiments, the third flexible electrically conductive material comprises any third flexible electrically conductive material described herein with respect to FIGS. 1A and 1B. In some embodiments, each contact pad is located within a contact pad well of the plurality of contact pad wells. In some embodiments, each contact pad is electrically coupled to a microelectrode lead of the plurality of microelectrode leads.

At 395, a second layer of PDMS is coated and cured on the flexible substrate. In some embodiments, the second layer of PDMS is coated via spin coating. In some embodiments, the second layer of PDMS electrically insulates each microelectrode lead, as well as one side of each microelectrode and one side of each contact pad, from the environment. In some embodiments, the electrical insulation of the second layer of PDMS ensures that the neural signals are not interfered with by undesired contact with the microelectrode leads or the “inactive” side of the contact pads or microelectrode leads.

In some embodiments, the method 300 further comprises etching the second layer of PDMS. In some embodiments, the second layer of PDMS is etched using a RIE process. In some embodiments, the RIE process is a SF₆ and O₂ RIE process. In some embodiments, etching the second layer of PDMS exposes the plurality of microelectrodes and the plurality of contact pads to the environment, while leaving the plurality of microelectrode leads electrically insulated from the environment. In some embodiments, this allows the plurality of microelectrodes to be electrically coupled to a single neuron or group of neurons and the plurality of contact pads to be electrically coupled to electronics that receive and process neural signals from the plurality of microelectrodes or that transmit neural signals to the plurality of microelectrodes.

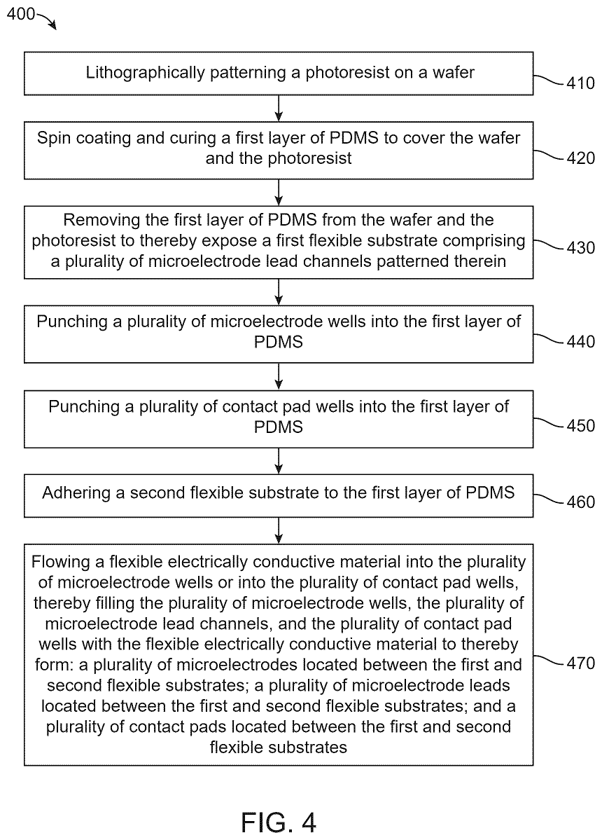

FIG. 4 shows a flowchart for a third method for manufacturing the exemplary flexible MEA system of FIGS. 1A and 1B. At 410, a photoresist is lithographically patterned on a wafer (such as a silicon wafer). In some embodiments, the photoresist comprises SU-8 photoresist. In some embodiments, the photoresist is patterned to form a partial negative mold of a portion of the microelectrode wells, the microelectrode lead channels, and a portion of the contact pad wells described herein.

At 420, a first layer of PDMS is coated and cured to cover the wafer and the photoresist. In some embodiments, the first layer of PDMS is coated via spin coating.

At 430, the first layer of PDMS is removed from the wafer and the photoresist to thereby expose a first flexible substrate. In some embodiments, the first flexible substrate comprises any flexible substrate described herein with respect to FIGS. 1A and 1B. In some embodiments, the first flexible substrate comprises a portion of any plurality of microelectrode wells, a plurality of microelectrode lead channels, and a portion of any plurality of contact pad wells described herein with respect to FIGS. 1A and 1B. In some embodiments, each microelectrode lead channel is coupled to a portion of a microelectrode well of the plurality of portions of the microelectrode wells. In some embodiments, each portion of each contact pad well is coupled to a microelectrode lead channel of the plurality of microelectrode lead channels.

At 440, a plurality of microelectrode wells is punched into the first layer of PDMS. In some embodiments, the punching transforms each portion of the plurality of microelectrode wells produced in steps 420 and 430 into a complete microelectrode well.

At 450, a plurality of contact pad wells is punched into the first layer of PDMS. In some embodiments, the punching transforms each portion of the plurality of contact pad wells produced in steps 420 and 430 into a complete contact pad well.

At 460, a second flexible substrate is adhered to the first layer of PDMS. In some embodiments, the second flexible substrate comprises any flexible substrate described herein with respect to FIGS. 1A and 1B. In some embodiments, each microelectrode well is coupled (for instance, mechanically or fluidically coupled) to a first end of a microelectrode lead channel of the plurality of microelectrode lead channels. In some embodiments, each contact pad well is coupled (for instance, mechanically or fluidically coupled) to a second end of the microelectrode lead channel. Thus, in some embodiments, the plurality of microelectrode wells, plurality of microelectrode lead channels, and plurality of contact pad wells form a plurality of flow paths between the first flexible substrate and the second flexible substrate. In some embodiments, one side of each microelectrode well and one side of each contact pad well are open to the environment, allowing a fluid to be flowed through the corresponding flow path.

At 470, a flexible electrically conductive material is flowed into the plurality of microelectrode wells or into the plurality of contact pad wells. In some embodiments, the flow fills the plurality of microelectrode wells, the plurality of microelectrode lead channels, and the plurality of contact pad wells with the flexible electrically conductive material. In some embodiments, this process forms the plurality of microelectrodes, the plurality of microelectrode leads, and the plurality of contact pads between the first and second flexible substrates. In some embodiments, the flexible electrically conductive material comprises any of the first, second, and third flexible electrically conductive materials described herein with respect to FIGS. 1A and 1B. In some embodiments, each microelectrode is located within a microelectrode well of the plurality of microelectrode wells. In some embodiments, each microelectrode lead is located within a microelectrode lead channel of the plurality of microelectrode channels. In some embodiments, each microelectrode lead is electrically coupled to a microelectrode of the plurality of microelectrodes. In some embodiments, each contact pad is located within a contact pad well of the plurality of contact pad wells. In some embodiments, each contact pad is electrically coupled to a microelectrode lead of the plurality of microelectrode leads.

In some embodiments, the method 400 further comprises etching the second flexible substrate. In some embodiments, the second flexible substrate is etched using a RIE process. In some embodiments, the RIE process is a SF₆ and O₂ RIE process. In some embodiments, etching the second layer of PDMS exposes the plurality of microelectrodes and the plurality of contact pads to the environment, while leaving the plurality of microelectrode leads electrically insulated from the environment. In some embodiments, this allows the plurality of microelectrodes to be electrically coupled to a single neuron or group of neurons and the plurality of contact pads to be electrically coupled to electronics that receive and process neural signals from the plurality of microelectrodes or that transmit neural signals to the plurality of microelectrodes.

### RECITATION OF EMBODIMENTS

Embodiment 1. A flexible microelectrode array system, comprising:
a flexible substrate comprising:
   a plurality of microelectrode wells patterned therein;
   a plurality of microelectrode lead channels patterned therein, each microelectrode lead channel coupled to a microelectrode well of the plurality of microelectrode wells; and
   a plurality of contact pad wells patterned therein, each contact pad well coupled to a microelectrode lead channel of the plurality of microelectrode lead channels;
a plurality of microelectrodes, each microelectrode located within a microelectrode well of the plurality of microelectrode wells, each microelectrode comprising a first flexible electrically conductive material;
a plurality of microelectrode leads, each microelectrode lead located within a microelectrode lead channel of the plurality of microelectrode lead channels, each microelectrode lead comprising a second flexible electrically conductive material, each microelectrode lead electrically coupled to a microelectrode of the plurality of microelectrodes; and
a plurality of contact pads, each contact pad located within a contact pad well of the plurality of contact pad wells, each contact pad comprising a third flexible electrically conductive material, each contact pad electrically coupled to a microelectrode lead of the plurality of microelectrode leads.

Embodiment 2. The system of Embodiment 1, wherein the flexible substrate is selected from the group consisting of: silicone, polydimethylsiloxane (PDMS), polyimide, and any combination thereof.

Embodiment 3. The system of Embodiment 1 or 2, wherein the first, second, and third flexible electrically conductive materials are the same.

Embodiment 4. The system of Embodiment 1 or 2, wherein the first, second, and third flexible electrically conductive materials are different.

Embodiment 5. The system of any one of Embodiments 1-4, wherein the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

Embodiment 6. A method for manufacturing a flexible microelectrode array system, comprising:
lithographically patterning a photoresist on a wafer;
coating and curing a first layer of PDMS to cover the wafer and the photoresist;
removing the first layer of PDMS from the wafer and the photoresist to thereby expose a flexible substrate comprising:
   a plurality of microelectrode wells patterned therein;
   a plurality of microelectrode lead channels patterned therein, each microelectrode lead channel coupled to a microelectrode well of the plurality of microelectrode wells; and
   a plurality of contact pad wells patterned therein, each contact pad well coupled to a microelectrode lead channel of the plurality of microelectrode lead channels;
filling the plurality of microelectrode wells with a first flexible electrically conductive material to thereby form a plurality of microelectrodes, each microelectrode located within a microelectrode well of the plurality of microelectrode wells;
filling the plurality of microelectrode lead channels with a second flexible electrically conductive material to thereby form a plurality of microelectrode leads, each microelectrode lead located within a microelectrode lead channel of the plurality of microelectrode channels, each microelectrode lead electrically coupled to a microelectrode of the plurality of microelectrodes;
filling the plurality of contact pad wells with a third flexible electrically conductive material to thereby form a plurality of contact pads, each contact pad located within a contact pad well of the plurality of contact pad wells, each contact pad electrically coupled to a microelectrode lead of the plurality of microelectrode leads; and
coating and curing a second layer of PDMS on the flexible substrate.

Embodiment 7. The method of Embodiment 6, wherein the first, second, and third flexible electrically conductive materials are the same.

Embodiment 8. The method of Embodiment 6, wherein the first, second, and third flexible electrically conductive materials are different.

Embodiment 9. The method of any one of Embodiments 6-8, wherein the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

Embodiment 10. The method of any one of Embodiments 6-9, further comprising etching the first layer of PDMS.

Embodiment 11. A method for manufacturing a flexible microelectrode array system, comprising:
lithographically patterning a first layer of photoresist on a wafer;
coating and curing a first layer of PDMS to cover the wafer and the first layer of photoresist;
lithographically patterning a second layer of photoresist on the first layer of PDMS;
selectively etching the first layer of PDMS through the second layer of photoresist;
removing the second layer of photoresist from the first layer of PDMS;
removing the first layer of PDMS from the wafer and the first layer of photoresist to thereby expose a flexible substrate comprising:
   a plurality of microelectrode wells patterned therein;
   a plurality of microelectrode lead channels patterned therein, each microelectrode lead channel coupled to a microelectrode well of the plurality of microelectrode wells; and
   a plurality of contact pad wells patterned therein, each contact pad well coupled to a microelectrode lead channel of the plurality of microelectrode lead channels;
filling the plurality of microelectrode wells with a first flexible electrically conductive material to thereby form a plurality of microelectrodes, each microelectrode located within a microelectrode well of the plurality of microelectrode wells;
filling the plurality of microelectrode lead channels with a second flexible electrically conductive material to thereby form a plurality of microelectrode leads, each microelectrode lead located within a microelectrode lead channel of the plurality of microelectrode channels, each microelectrode lead electrically coupled to a microelectrode of the plurality of microelectrodes;
filling the plurality of contact pad wells with a third flexible electrically conductive material to thereby form a plurality of contact pads, each contact pad located within a contact pad well of the plurality of contact pad wells, each contact pad electrically coupled to a microelectrode lead of the plurality of microelectrode leads; and
coating and curing a second layer of PDMS on the flexible substrate.

Embodiment 12. The method of Embodiment 11, wherein the first, second, and third flexible electrically conductive materials are the same.

Embodiment 13. The method of Embodiment 11, wherein the first, second, and third flexible electrically conductive materials are different.

Embodiment 14. The method of any one of Embodiments 11-13, wherein the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

Embodiment 15. The method of any one of Embodiments 11-14, further comprising etching the second layer of PDMS.

Embodiment 17. A method for manufacturing a flexible microelectrode array system, comprising:
lithographically patterning a photoresist on a wafer;
coating and curing a first layer of PDMS to cover the wafer and the photoresist;
removing the first layer of PDMS from the wafer and the photoresist to thereby expose a first flexible substrate comprising a plurality of microelectrode lead channels patterned therein;
punching a plurality of microelectrode wells into the first layer of PDMS, each microelectrode coupled to a first end of a microelectrode lead channel of the plurality of microelectrode lead channels;
punching a plurality of contact pad wells into the first layer of PDMS, each contact pad well coupled to a second end of a microelectrode lead channel of the plurality of microelectrode lead channels;
adhering a second flexible substrate to the first layer of PDMS; and
flowing a flexible electrically conductive material into the plurality of microelectrode wells or into the plurality of contact pad wells, thereby filling the plurality of microelectrode wells, the plurality of microelectrode lead channels, and the plurality of contact pad wells with the flexible electrically conductive material to thereby form:
   a plurality of microelectrodes located between the first and second flexible substrates, each microelectrode located within a microelectrode well of the plurality of microelectrode wells;
   a plurality of microelectrode leads located between the first and second flexible substrates, each microelectrode lead located within a microelectrode lead channel of the plurality of microelectrode channels, each microelectrode lead electrically coupled to a microelectrode of the plurality of microelectrodes; and
   a plurality of contact pads located between the first and second flexible substrates, each contact pad located within a contact pad well of the plurality of contact pad wells, each contact pad electrically coupled to a microelectrode lead of the plurality of microelectrode leads.

Embodiment 17. The method of Embodiment 16, wherein the second flexible substrate is selected from the group consisting of: silicone, PDMS, polyimide, and any combination thereof.

Embodiment 18. The method of Embodiment 16 or 17, wherein the flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

1. A flexible microelectrode array system, comprising:
a flexible substrate comprising:
a plurality of microelectrode wells patterned therein;
a plurality of microelectrode lead channels patterned therein, each microelectrode lead channel coupled to a microelectrode well of the plurality of microelectrode wells; and
a plurality of contact pad wells patterned therein, each contact pad well coupled to a microelectrode lead channel of the plurality of microelectrode lead channels;
a plurality of microelectrodes, each microelectrode located within a microelectrode well of the plurality of microelectrode wells, each microelectrode comprising a first flexible electrically conductive material;
a plurality of microelectrode leads, each microelectrode lead located within a microelectrode lead channel of the plurality of microelectrode lead channels, each microelectrode lead comprising a second flexible electrically conductive material, each microelectrode lead electrically coupled to a microelectrode of the plurality of microelectrodes; and
a plurality of contact pads, each contact pad located within a contact pad well of the plurality of contact pad wells, each contact pad comprising a third flexible electrically conductive material, each contact pad electrically coupled to a microelectrode lead of the plurality of microelectrode leads.

2. The system of claim 1, wherein the flexible substrate is selected from the group consisting of: silicone, polydimethylsiloxane (PDMS), polyimide, and any combination thereof.

3. The system of claim 1, wherein the first, second, and third flexible electrically conductive materials are the same.

4. The system of claim 1, wherein the first, second, and third flexible electrically conductive materials are different.

5. The system of claim 1, wherein the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

6. A method for manufacturing a flexible microelectrode array system, comprising:
lithographically patterning a photoresist on a wafer;
coating and curing a first layer of PDMS to cover the wafer and the photoresist;
removing the first layer of PDMS from the wafer and the photoresist to thereby expose a flexible substrate comprising:
a plurality of microelectrode wells patterned therein;
a plurality of microelectrode lead channels patterned therein, each microelectrode lead channel coupled to a microelectrode well of the plurality of microelectrode wells; and
a plurality of contact pad wells patterned therein, each contact pad well coupled to a microelectrode lead channel of the plurality of microelectrode lead channels;
filling the plurality of microelectrode wells with a first flexible electrically conductive material to thereby form a plurality of microelectrodes, each microelectrode located within a microelectrode well of the plurality of microelectrode wells;
filling the plurality of microelectrode lead channels with a second flexible electrically conductive material to thereby form a plurality of microelectrode leads, each microelectrode lead located within a microelectrode lead channel of the plurality of microelectrode channels, each microelectrode lead electrically coupled to a microelectrode of the plurality of microelectrodes;
filling the plurality of contact pad wells with a third flexible electrically conductive material to thereby form a plurality of contact pads, each contact pad located within a contact pad well of the plurality of contact pad wells, each contact pad electrically coupled to a microelectrode lead of the plurality of microelectrode leads; and
coating and curing a second layer of PDMS on the flexible substrate.

7. The method of claim 6, wherein the first, second, and third flexible electrically conductive materials are the same.

8. The method of claim 6, wherein the first, second, and third flexible electrically conductive materials are different.

9. The method of claim 6, wherein the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

10. The method of claim 6, further comprising etching the first layer of PDMS.

11. A method for manufacturing a flexible microelectrode array system, comprising:
lithographically patterning a first layer of photoresist on a wafer;
coating and curing a first layer of PDMS to cover the wafer and the first layer of photoresist;
lithographically patterning a second layer of photoresist on the first layer of PDMS;
selectively etching the first layer of PDMS through the second layer of photoresist;
removing the second layer of photoresist from the first layer of PDMS;
removing the first layer of PDMS from the wafer and the first layer of photoresist to thereby expose a flexible substrate comprising:
a plurality of microelectrode wells patterned therein;
a plurality of microelectrode lead channels patterned therein, each microelectrode lead channel coupled to a microelectrode well of the plurality of microelectrode wells; and
a plurality of contact pad wells patterned therein, each contact pad well coupled to a microelectrode lead channel of the plurality of microelectrode lead channels;
filling the plurality of microelectrode wells with a first flexible electrically conductive material to thereby form a plurality of microelectrodes, each microelectrode located within a microelectrode well of the plurality of microelectrode wells;
filling the plurality of microelectrode lead channels with a second flexible electrically conductive material to thereby form a plurality of microelectrode leads, each microelectrode lead located within a microelectrode lead channel of the plurality of microelectrode channels, each microelectrode lead electrically coupled to a microelectrode of the plurality of microelectrodes;
filling the plurality of contact pad wells with a third flexible electrically conductive material to thereby form a plurality of contact pads, each contact pad located within a contact pad well of the plurality of contact pad wells, each contact pad electrically coupled to a microelectrode lead of the plurality of microelectrode leads; and
coating and curing a second layer of PDMS on the flexible substrate.

12. The method of claim 11, wherein the first, second, and third flexible electrically conductive materials are the same.

13. The method of claim 11, wherein the first, second, and third flexible electrically conductive materials are different.

14. The method of claim 11, wherein the first, second, or third flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.

15. The method of claim 11, further comprising etching the second layer of PDMS.

16. A method for manufacturing a flexible microelectrode array system, comprising:
lithographically patterning a photoresist on a wafer;
coating and curing a first layer of PDMS to cover the wafer and the photoresist;
removing the first layer of PDMS from the wafer and the photoresist to thereby expose a first flexible substrate comprising a plurality of microelectrode lead channels patterned therein;
punching a plurality of microelectrode wells into the first layer of PDMS, each microelectrode coupled to a first end of a microelectrode lead channel of the plurality of microelectrode lead channels;
punching a plurality of contact pad wells into the first layer of PDMS, each contact pad well coupled to a second end of a microelectrode lead channel of the plurality of microelectrode lead channels;
adhering a second flexible substrate to the first layer of PDMS; and
flowing a flexible electrically conductive material into the plurality of microelectrode wells or into the plurality of contact pad wells, thereby filling the plurality of microelectrode wells, the plurality of microelectrode lead channels, and the plurality of contact pad wells with the flexible electrically conductive material to thereby form:
a plurality of microelectrodes located between the first and second flexible substrates, each microelectrode located within a microelectrode well of the plurality of microelectrode wells;
a plurality of microelectrode leads located between the first and second flexible substrates, each microelectrode lead located within a microelectrode lead channel of the plurality of microelectrode channels, each microelectrode lead electrically coupled to a microelectrode of the plurality of microelectrodes; and
a plurality of contact pads located between the first and second flexible substrates, each contact pad located within a contact pad well of the plurality of contact pad wells, each contact pad electrically coupled to a microelectrode lead of the plurality of microelectrode leads.

17. The method of claim 16, wherein the second flexible substrate is selected from the group consisting of: silicone, PDMS, polyimide, and any combination thereof.

18. The method of claim 16, wherein the flexible electrically conductive material is selected from the group consisting of: a mixture of silicone and carbon nanotubes (CNTs), a mixture of PDMS and CNTs, CNT ink, a metallic ink, silver ink, gold ink, aluminum ink, copper ink, and any combination thereof.
